(19) United States
(12) Patent Application Publication
(10) Pub. No.: US 2017/0143862 A1
(43) Pub. Date: May 25, 2017

(54) DEODORANT

(71) Applicant: Ishizuka Glass Co., Ltd., Iwakura-Shi (JP)

(72) Inventor: Ayako ISHIKAWA, Iwakura-Shi (JP)

(21) Appl. No.: 14/945,894

(22) Filed: Nov. 19, 2015

(51) Int. Cl. A61L 9/012

(52) U.S. Cl. CPC A61L 9/012

(57) ABSTRACT

There is provided a deodorant which can stably exhibit deodorizing effect for a long term, does not agglomerate even when used in a powder form, has a high degree of freedom in product form and use mode, and is highly convenient, as compared with the prior art. The deodorant comprises a glass, the glass containing: 46 to 70 mol % of SiO₂; 15 to 50 mol % of B₂O₃ and R₂O (R=Li, Na, K) in total; 0 to 10 mol % of R′O (R′=Mg, Ca, Sr, Ba); and 0.1 to 23 mol % of CuO.

### BACKGROUND

1. Technical Field

The present invention relates to a deodorant having the function of deodorizing sulfur-based offensive odor substances such as hydrogen sulfide and methyl mercaptan and other odor substances such as lower fatty acids and body odor components.

2. Related Art

In recent years, the demand for various kinds of deodorants is rising, in response to increase in interest in a comfortable living environment.

Among odors which are problematic in a living environment, sulfur-based offensive odors emitted from hydrogen sulfide, methyl mercaptan and the like are hated as those which give a strong discomfort. Especially, methyl mercaptan is known as an offensive odor-causing substance which emits a putrid odor even at a low level in a ppb order, and the technical development concerning deodorization thereof has conventionally been demanded.

As the above-described technique concerning deodorization, there are disclosed the technique of incorporating any of silver, copper and iron in a soluble glass mainly containing P₂O₅ and setting the speed of dissolution of PO₄²⁻ ions, Ag⁺ ions, Cu²⁺ ions and Fe²⁺ ions within specific ranges, thereby deodorizing sulfur-based offensive odors (JP 1992-67868 A) and the technique of removing odor causing substances such as methyl mercaptan by a deodorant obtained by dispersing copper oxide in activated carbon (JP 2009-213992 A).

However, the technique disclosed in JP 1992-67868 A involves the following problems. This technique utilizes a sulfurization reaction between sulfur components and Ag⁺ ions, Cu²⁺ ions and Fe²⁺ ions produced in dissolution, and thus, when they are in an equilibrium state, the reaction does not proceed any more so that no sustainable deodorizing effect can be expected. Also, a soluble glass agent mainly containing P₂O₅ lacks chemical durability, especially, water resistance. Therefore, when the glass is used in a powder form, it easily agglomerates and thus becomes difficult to handle, and is otherwise restricted in terms of product form, use mode, etc., and thus is poor in convenience.

JP 2009-213992 A fails to describe a specific action of copper oxide, but it is assumed that the odor substance removing efficiency of activated carbon would be improved by its catalytic action. However, the technique disclosed in JP 2009-213992 A involves the problem that copper oxide dispersed in activated carbon is poisoned (catalyst degradation) by reactions with odor causing substances so that the duration of the deodorizing effect still remains unsatisfactory.

### PRIOR ART DOCUMENT

[Patent Document 1] JP 1992-67868 A
[Patent Document 2] JP 2009-213992 A

### SUMMARY

An object of the present invention is to solve the problems as raised above and to provide a deodorant which can stably exhibit deodorizing effect for a long term, does not agglomerate even when used in a powder form, has a high degree of freedom in product form and use mode, and is highly convenient, as compared with the prior art.

The deodorant of the present invention which has been made to solve the above problems is preferably a deodorant comprising a glass, the glass containing: 46 to 70 mol % of SiO₂; 15 to 50 mol % of B₂O₃ and R₂O (R=Li, Na, K) in total; 0 to 10 mol % of R′O (R′=Mg, Ca, Sr, Ba); and 0.1 to 23 mol % of CuO. The above-described glass preferably contains 5 to 20 mol % of B₂O₃; and 10 to 30 mol % of R₂O (R=Li, Na, K).

The glass composition as described above preferably contains: 50 to 63 mol % of SiO₂; 23 to 44 mol % of B₂O₃ and R₂O (R=Li, Na, K) in total; 2 to 7 mol % of R′O (R′=Mg, Ca, Sr, Ba); and 1 to 13 mol % of CuO, and, further, more preferably contains 8 to 18 mol % of B₂O₃; and 15 to 26 mol % of R₂O (R=Li, Na, K).

The glass composition as described above preferably contains: 51 to 55 mol % of SiO₂; 12 to 16 mol % of B₂O₃; 19 to 22 mol % of Na₂O; 4.5 to 6.5 mol % of CaO; and 4 to 13 mol % of CuO.

A deodorant which has a high degree of freedom in product form and use mode and is highly convenient as compared with the prior art can be realized by using, as a deodorant, glass having the above-described composition containing 5 to 20 mol % of B₂O₃ and 10 to 30 mol % of R₂O (R=Li, Na, K). Particularly, there can be realized a deodorant which can stably exhibit deodorizing effect for a long term, has high chemical durability, is hard to agglomerate even when used in a powder form, can exhibit an excellent deodorizing effect even at room temperature and in the presence of oxygen, in the dark without light, in the presence of moisture (in a state of wetted surface), or in a high-temperature environment (450° C. or lower), and which is quite easy to handle.

Conventionally, there has existed no “glass agent which exhibits deodorizing effect due to the catalytic action,” and various deodorants using a soluble glass have exclusively been developed. On the other hand, as a result of long-term researches, the present inventors have newly found that “CuO contained in the above-described proportion in the glass having the above-described composition functions as a catalyst, promotes decomposition reactions (oxidation/reduction reactions) of sulfur-based offensive odor substances, and provides the effect of deodorizing such sulfur-based offensive odor substances.” The present invention has been made based on this finding, and is expected to be developed to various applications as a “novel glass agent which exhibits deodorizing effect due to the catalytic action.”

Since the present invention has a mechanism of using CuO contained in glass as a catalyst to promote decomposition reactions of sulfur-based offensive odor substances, it is possible to increase the deodorization capacity (which is proportional to the concentration of ions which adsorb the offensive odor component of a sulfur component, for example, in JP 1992-67868 A) and to maintain the deodorizing effect over a long term by using the catalyst repeatedly. Also, poisoning, as in the prior art in which CuO functioning as a catalyst and is dispersed in activated carbon, hardly proceeds (for example, JP 2009-213992 A), and thus CuO can stably exhibit its catalytic function over a long term.

The deodorant of the present invention can exhibit an excellent deodorizing effect, especially, on methyl mercaptan. Incidentally, the deodorant is used in a powder form to ensure a large contact area with odor substances, and thus can more effectively function as a catalyst.

The deodorant of the present invention can deodorize any odor substances that can cause a dehydrogenation reaction, not limited to sulfur-based offensive odor substances. Particularly, examples of odor substances which can be deodorized include lower fatty acids and acetic acid and isovaleric acid which are known as body odors (sweat and food odor) as well as propionic acid and normal-butyric acid and normal-valeric acid designated by the Offensive Odor Control Law; mid-chain fatty acids such as caproic acid and enanthic acid; and trans-2-nonenal which is known as an unpleasant body odor of old people. In general, fatty acids having 2 to 4 carbon atoms refer to short-chain fatty acids (lower fatty acids), but acetic acid having 1 carbon atom and valeric acid having 5 carbon atoms are also treated as lower fatty acids herein. There is a high possibility that the mechanism of deodorizing these lower fatty acids and trans-2-nonenal may be similar to the catalytic action on sulfur-based offensive odors. For example, while the deodorant of the present invention catalytically decomposes methyl mercaptan to produce a dimer dimethyl disulfide, a dehydrogenation reaction would take place at this time. Similarly, lower fatty acids are assumed to be decomposed by a dehydrogenation reaction. Or, malodorous gases generated by lower fatty acids are known to be acidic, and thus may cause a neutralization reaction with the deodorant of the present invention containing a large amount of an alkali. When the reaction amount was calculated from deodorization test results, the deodorizing effect equal to or higher than that obtained in an equivalent amount reaction was confirmed. So, there is a high possibility that the deodorizing effect due to the catalytic action and the deodorizing effect due to the neutralization reaction may concurrently be produced. However, trans-2-nonenal is known as a neutral gas, and thus there is a high possibility that the deodorizing effect thereon may be mainly the deodorizing effect due to the catalytic action, not the neutralization reaction. It is also considered that the deodorant decomposes not only trans-2-nonenal but also its precursor palmitoleic acid to provide deodorizing effect.

Also, the deodorant of the present invention contains CuO in a large amount in glass, and thus can also provide antibacterial effect simultaneously.

Other conventional techniques utilizing a “sulfurization reaction” (for example, a deodorizing method comprising reacting sulfur components with Ag⁺ ions, Cu²⁺ ions and Fe²⁺ ions having high affinity for the sulfur components in JP 1992-67868 A etc.) also involve the problem that the sulfurization reaction causes discoloration of glass, leading to impaired aesthetic appearance of the glass. On the other hand, the present invention is intended to promote the decomposition reaction of sulfur-based offensive substances using vitrified CuO as a catalyst to provide the sulfur-based offensive substance deodorizing effect, and thus can provide deodorizing function without discoloration of glass.

### BRIEF DESCRIPTION OF DRAWINGS

FIG. 1 is a graph showing the measurement result of Example A.

FIG. 2 is a graph showing the measurement result of Example B.

FIG. 3 is a graph showing the measurement result of Example B.

FIG. 4 is a graph showing the measurement result of Example C.

FIG. 5 is a graph showing the measurement result of Example D.

FIG. 6 is a graph showing the measurement result of Example E.

FIG. 7 is a graph showing the measurement result of Example G.

FIG. 8 is a graph showing the measurement result of Example G.

FIG. 9 is a graph showing the measurement result of Example H.

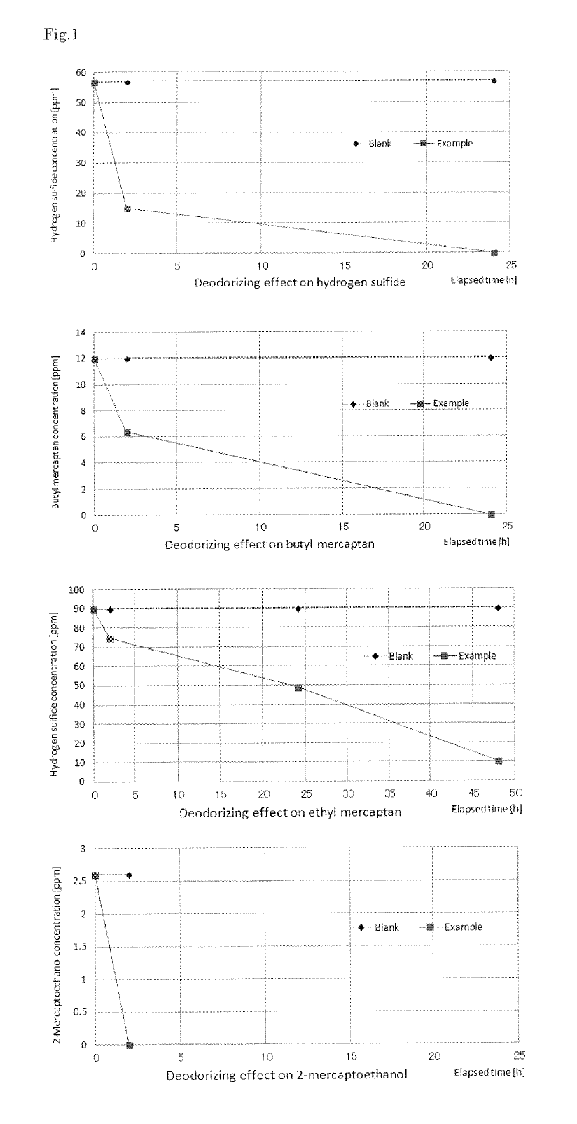

### DETAILED DESCRIPTION

Hereinafter, preferred embodiments of the present invention are described.

The deodorant of the present embodiment comprises “alkali (R₂O)-alkali earth (R′O)-borosilicate glass (B₂O₃—SiO₂)” containing 46 to 70 mol % of SiO₂; 15 to 50 mol % of B₂O₃ and R₂O in total; 0 to 10 mol % of R′O (R′=Mg, Ca, Sr, Ba); and 0.1 to 23 mol % of CuO, and can be produced by the melt quenching method, like normal glass agents. The form of the glass agent is not particularly limited, but can be, for example, a molded body or a powder. In the case of a molded body (for example, a deodorization container), molds are used to mold a molded body. In the case of a powder, a pre-molded body is obtained by the melt quenching method and then pulverized, whereby a deodorant having a desired form can be obtained. Pulverization as used herein means pulverization by using a generally known pulverizer (for example, a ball mill, a bead mill, a jet mill or a CF mill), which may be either dry or wet.

Hereinafter, the respective glass components will be explained in detail.

### (SiO₂)

SiO₂ serves as a main component which forms the glass-network. The content of SiO₂ is 46 to 70 mol %, preferably 50 to 63 mol %. A content of less than 46 mol % is not preferred since the chemical durability of the glass becomes insufficient and the glass easily devitrify. Further, a content of less than 46 mol % is not preferred since the water resistance of the glass becomes insufficient and copper ions are easily eluted in the presence of moisture (including moisture in the atmosphere), resulting in a stronger deodorizing effect due to the sulfurization reaction caused by ion elution than the deodorizing effect due to the catalytic action. A content of more than 70 mol % is not preferred since the glass becomes hard to melt due to its elevated melting point and, additionally, has increased viscosity.

(B₂O₃)

B₂O₃ is a component which improves glass solubility and clarity, and also becomes a component which forms the glass-network in a specific composition. B₂O₃ greatly affects the stability of the glass depending on its content, and plays a great role as a flux in the present invention. The content is 5 to 20 mol %, preferably 8 to 18 mol % in view of the amount of B₂O₃ to be volatilized. A content of more than 20 mol % is not preferred since B₂O₃ is easily volatilized in the melting process so that the composition is hard to control.

### (R₂O (R=Li, Na, K))

R₂O (R=Li, Na, K) is a component which cuts a bond between Si and O in the network of glass to form non-crosslinking oxygen, and, as a result, reduces the viscosity of the glass and improves the moldability and solubility thereof, and is a flux like B₂O₃. The total content of one or two or more of R₂O (R=Li, Na, K) is defined as 10 to 30 mol %, preferably 15 to 26 mol % also in view of the content ratio thereof to the other components. When the content is more than 30 mol %, the chemical durability of the glass becomes insufficient. Specifically, the glass agent is reacted with moisture in the atmosphere to cause a whitening phenomenon called bloom. The generation of bloom undesirably reduces the contact area with malodorous gases. Also, the alumina matter in a melting furnace is easily eroded.

(B₂O₃+R₂O (R=Li, Na, K))

Both of B₂O₃ and R₂O are used as fluxes, as described above. A range of the total content of B₂O₃ and R₂O of 15 to 50 mol %, preferably 23 to 44 mol % is a region where the deodorizing effect is safely exhibited. A content of less than 15 mol % is not preferred since the solubility of the glass becomes insufficient and the glass easily devitrify during molding. A content of more than 45 mol % is not preferred since the water resistance of the glass becomes insufficient and copper ions are easily eluted in the presence of moisture (including moisture in the atmosphere), resulting in a stronger deodorizing effect due to the sulfurization reaction caused by ion elution than the deodorizing effect due to the catalytic action. Also, a content of more than 50 mol % is not preferred since phase splitting easily occurs during melting, resulting in an insufficient deodorizing effect of the glass agent.

### (R′O (R′=Mg, Ca, Sr, Ba))

R′O (R′=Mg, Ca, Sr, Ba) is a component which improves the chemical durability of glass. The total content of one or two or more of R′O (R′=Mg, Ca, Sr, Ba) is defined as 0 to 10 mol %, preferably 2 to 7 mol %. A content of more than 10 mol % is not preferred since the glass has increased viscosity during melting and easily loses its transparency. Incidentally, this is not an essential component for the deodorant of the present invention, and the content thereof may be 0 mol %.

### (CuO)

CuO functions as a catalyst, promotes decomposition reactions (oxidation/reduction reactions) of sulfur-based offensive odor substances, and provides the effect of deodorizing sulfur-based offensive odor substances. The content of CuO is 0.1 to 23 mol %, preferably 1 to 13 mol %, more preferably 4 to 13 mol %. A content of more than 23 mol % is not preferred since unmolten products easily remain and, additionally, metal copper is easily deposited during quenching or processing. Since metal copper also exhibits deodorizing effect, the deposition thereof causes no problem from the viewpoint of deodorization. However, since glass is discolored along with the deposition of metal copper, the deposition is not suitable for applications in which discoloration of glass causes a problem. Also, in the case where the deposition of metal copper causes, poisoning. Contrary to this, according to the present invention in which CuO is incorporated as a glass component, poisoning is hard to proceed so that it can stably exert the catalyst function over a long term.

When the content of CuO is gradually decreased under the conditions that glass agents have the same weight and particle size, the deodorizing ability tends to deteriorate along with the decrease of the CuO content. This is assumed to be caused by reduction in the amount of CuO on the glass surface which is contacted with offensive odor. The CuO content and particle size vary depending on the required deodorizing speed and/or deodorization capacity, but the particle size is defined as D50 (corresponding to 50% integrated value when the particle size is cumulatively distributed, referred to generally as median diameter)=0.1 m or more, preferably D50=1 m or more, more preferably D50=4 pin or more, from the viewpoint of the productivity and production cost. When the particle size is defined as D50=0.1 μm or more, the content of CuO is defined as 0.1 mol % or more. When the particle size is defined as D50=1 μm or more, the content of CuO is defined as 1 mol % or more. When the particle size is defined as D50=4 μm or more, the content of CuO is defined as 4 mol % or more.

As regards the CuO content and particle size, the surface area per unit mass of the powder is referred to as specific surface area [m²/g], and the larger this value is, the finer the particles are. Assuming that the particles are in a spherical form, when there are n particles having a radius r, the total surface area at this time is n4πr², and, when ρ denotes the density of the particles, the mass is (n4πr³/3)ρ. Thus, the specific surface area=n4πr²/(n4πr³/3)ρ=3/ρr. Here, assuming that the radius of the deodorant glass agent particles is R and that the density is P, the specific surface area is expressed as 3/PR. When R=5 μm, the specific surface area (diameter=2R=10 μm)=3/P (5 μm), and, when 0.5 μm, the specific surface area (diameter=2R=1 μm)=3/P (0.5 μm). Specifically, when the particle size (diameter) of the glass agent, 10 μm, is decreased down to 1 μm, the specific surface area becomes 10 times larger. According to this, the deodorizing ability is assumed to become higher, of course. In view of the above, the amount of CuO to be added can be extremely decreased if the particle size can be made small. However, in order to obtain a stable deodorizing ability, desirably, the CuO content is 0.1 mol % or more and the particle size is 0.1 μm or more, in view of the control during production, productivity and production price.

In the present invention in which CuO is incorporated as a glass component, copper ions, which are transition metal ions, have been introduced into the matrix of glass. Copper ions are known to be strongly affected by the crystal field from circumferential negative ions, when introduced into the glass matrix. Copper ions take a plurality of ion states depending on the surrounding environment, but normally exist as Cu⁺ or Cu²⁺ in glass. Cu²⁺ is stable in an oxidation atmosphere, and Cu⁺ is stable in a reducing atmosphere. Cu²⁺ in the glass occupies the position of network modification ions, and develops blue color when many oxygen ions are coordinated with this. Cu⁺ itself is colorless, but, when it coexists with Cu²⁺, the ions are deformed, resulting in enhanced absorption. Also, when the copper ion concentration is increased, it becomes impossible to satisfy oxygen ion coordination with all of Cu²⁺, resulting in an increased number of unsaturated copper ions having a low coordination number. Also, unsaturated ions are increased by temperature rise. Along with this, the glass changes its color from blue to green. Cu²⁺ shows an absorption band in a range from the visible region to the near-infrared region (around 800 nm). In general, examples of the factor for determining the atomic value of the transition metal ions include melting temperature, oxygen partial pressure in a melting atmosphere, amount of transition metal ions to be added, and host glass composition. However, there are only a few reports on the atomic value regulation of copper ions by the glass composition.

It is known that the addition of alumina to oxide glass improves the water resistance of the glass. For example, according to the studies made by Murata, Kurimura, Morinaga et al. (J. Japan Inst. Met. Mater., Vol. 61, No. 11 (1997)), the following matter has been confirmed in a specific composition. In general, silicate-based glass has a higher melting temperature than borate- or phosphate-based glass, and thus the oxidation/reduction state of Cu⁺-Cu²⁺ is apt to transfer to the reduction side, as compared with the other two glass systems. The addition of alumina to borate- or phosphate-based glass provides the effect of stabilizing the oxidation/reduction state of Cu⁺-Cu²⁺ to the reduction side. It has been reported that, in two-component Na₂O—SiO₂ glass, Cu⁺ relatively increases along with the reduction in Na₂O content, and that, in three-component alkali-alkali earth-silicate glass, the amount of Cu⁺ increases along with the decrease in ion radius of the alkali earth. Also, there is a report that copper ions, among transition metals, are special in the way of influence on the valence balance by the host glass. However, the actions exerted by the respective components of the glass agent do not always change linearly in accordance with their blending proportions. Various factors such as atomic bonds and change in binding nuclei in the amorphous and vitreous agent are considered to act on this.

(Al₂O₃)

Al₂O₃ is a component which improves the chemical durability of glass and affects the stability of the crystal structure. Also, Al2O3 functions to suppress phase separation of glass and to enhance the homogeneity of the glass agent. Since there is a possibility that the oxidation/reduction state of copper ions in the glass may be affected by increase of the viscosity or addition, the content of Al₂O₃ is desirably 3.5 mol % or less.

When the amount of CuO to be added exceeds 23 mol %, copper ions are reduced during quenching or molding after glass melting so that metal copper is deposited, in some cases. Metal copper also exhibits deodorizing effect, and thus the deposition thereof causes no problem from the viewpoint of deodorization. However, when CuO is deposited as metal copper, poisoning would proceed. At this time, the deposition of metal copper can be suppressed by employing Al³⁺ as a part of the glass structure composed of SiO₂.

### (Other Minor Components)

In addition to the above components, ZnO, SrO, BaO, TiO₂, ZrO₂, Nb₂O₅, P₂O₅, Cs₂O, Rb₂O, TeO₂, BeO, GeO₂, Bi₂O₃, La₂O₃, Y₂O₃, WO₃, MoO₃, Fe₂O₃ or the like can also be incorporated as a minor component. Further, F, Cl, SO₃, Sb₂O₃, SnO₂, Ce or the like may be added as a clarifying agent.

(Fe₂O₃)

Fe₂O₃ is a component which affects the oxidation/reduction state of copper ions in glass (reinforces Cu⁺>Cu²⁺), and thus the content there of is desirably 0.2 mol % or less, preferably 0.1 mol % or less.

(Cr₂O₃, MnO₂, CeO₂)

Cr₂O₃, MnO₂ and CeO₂ are transition metal ions, and are components which can change the atomic value like CuO. When these components are mixed with CuO, the oxidation/reduction state of copper ions in glass leans towards acidity (Cu⁺<Cu²⁺) due to these components with strong oxidizability (oxidation power: Cr₂O₃>MnO₂>CeO₂). The deodorizing effect can stably be obtained by adopting the composition range and production method of the present invention, but, when the expectation on the oxidation/reduction state is greatly wrong so that no deodorizing effect can be obtained (for example, the melting furnace may sometimes be difficult to regulate the oxidation/reduction state along with erosion), the valence balance of the copper ions can also be regulated by addition of Cr₂O₃, MnO₂ and/or CeO₂.

In view of the above, the composition range which stably provides the deodorizing effect has been specified in the present invention. Specifically, the composition range has been specified in consideration of the melting temperature range, oxidation/reduction state and composition range. A deodorant glass agent can stably be obtained by producing a glass agent falling within the above-described composition range by the melt quenching method. Especially, such a glass agent can stably be obtained by tank furnace melting, electric furnace melting or small-scale crucible melting. In general, in the case of soda lime glass, the tank furnace melting and electric furnace melting are known to provide a valence balance of copper ions (Cu²⁺/total) of about 15% and about 50%, respectively. The valence balance is naturally changed also by the composition of the present invention. The valence balance naturally changes also in the case of the composition of the present invention. Since the deodorizing mechanism is catalytic action, the chemical states of these may affect the deodorizing effect, but their difference in effect does not especially cause a problem if they fall within the above-described composition range.

The fact that the oxidation/reduction state varies depending on the melting temperature and melting time must be considered. The melting temperature is preferably controlled within the range of 1200 to 1400° C., preferably 1280° C. to 1380° C. The melting time is desirably 6 to 8 hours. The glass obtained herein is confirmed to develop blue color or blue green color due to Cu²⁺. The valence balance of copper ions is not always important within the composition range of the present invention, as described above. When the glass agents obtained were intentionally changed in valence balance (thin plates were prepared; blue glass confirmed to develop a color of Cu²⁺, glass changed in valence balance to Cu⁺>>Cu²⁺ and confirmed to hardly have a color hue, and brown (red) glass confirmed to have deposited colloidal metal copper of Cu⁰) to confirm their deodorizing effect, a sufficient deodorizing effect was obtained in each case. Thus, glass agents falling within the above-described composition range can provide deodorizing effect, and the deodorizing effect can also be obtained by regulating the valence balance of copper ions, for example, through heat treatment after molding.

The form of the glass agent is not particularly limited as described above, and the glass agent can be used as is as a deodorant product put in a powder or granular form in a container such as a cartridge, and, additionally, can impart deodorizing performance to fibers, coating materials, sheets, molded articles and the like, and can be used as a deodorant product. The use form is not limited to powder, and may be either plate or molded body. The deodorant based on the catalytic action may sometimes be insufficient in immediate effect when the offensive odor concentration is high. The deodorant can also be mixed with a physical adsorbent (activated carbon, silica gel, zeolite or the like) as a temporary trapping agent for use. Also, since odor is not always present as one-component odor, agents specialized in deodorization of various offensive odors can also be utilized in combination. The deodorant can also be mixed with a conventional deodorant for use.

### EXAMPLES

### Method for Preparing Deodorant Glass Agent:

After blending of raw materials, the blend was molten at a melting temperature of 1350° C. for 8 hours and poured out, thereby producing glass having the glass composition indicated in Table 1. After melting, the glass was naturally cooled, but may also be water-cooled. The glass composition was confirmed by semi-quantitative measurement using a fluorescence X-ray analyzer. The resultant glass was dry-pulverized in a ball mill and regulated so that D50=4.5 μm or less and D98 (corresponding to 98% integrated value when the particle size is cumulatively distributed)=50 μm or less by means of a particle size meter. The particles having a particle size (diameter) of 100 μm or more were removed by sieving.

TABLE 1
Compositional ratio of deodorant glass agent (mol %)

| | Example |
|---|---|
| B₂O₃ | 13.6 |
| SiO₂ | 52.5 |
| CaO | 5.6 |
| Na₂O | 20.4 |
| CuO | 7.9 |
| Total amount of CuO in 0.1 g of sample [mol] | 1.26 × 10⁻⁴ |
| Specific surface area [m²/g] | 1.54 |
| Particle size (D50) | 4.21 |

### Example A: Test for Confirming Deodorizing Effect on Sulfur-Based Offensive Odor

### Deodorization Test Method:

The deodorant glass agent having the glass composition indicated in Table 1 and offensive odor were enclosed in a Tedlar bag to measure the offensive odor concentration in the bag in accordance with elapsed time by means of a gas detecting tube.

The test conditions were defined as follows.

Tedlar bag capacity: 1 L
Temperature: room temperature (20 to 25° C.)
Weight of deodorant glass agent: 0.1 g
Particle size of deodorant glass agent: D50=4.21 μm
Specific surface area of deodorant glass agent: 1.54 m²/g

Operations similar to the above operations were conducted without the deodorant glass agent as a blank.

### Measurement Result and Consideration:

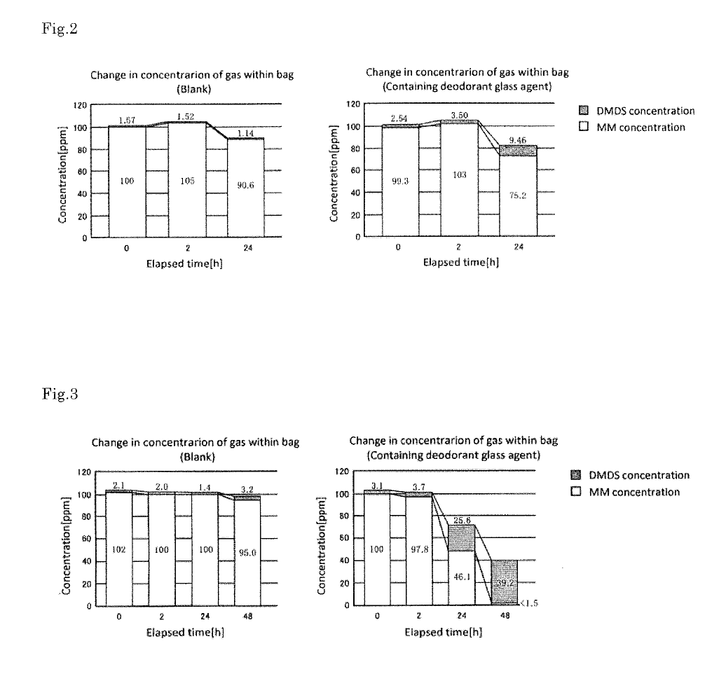

It was confirmed that the deodorant glass agent has deodorizing effect on hydrogen sulfide, ethyl mercaptan, butyl mercaptan, 2-mercaptoethanol and any sulfur-based offensive odors. Additionally, the deodorant glass agent was confirmed to have deodorizing effect also on methyl mercaptan as shown in FIGS. 2, 3, 4 and 6.

### Example B: Test for Elucidating Deodorizing Mechanism of Deodorant Glass Agent

### Deodorization Test Method 1 (Nitrogen Atmosphere):

The deodorant glass agent having the glass composition indicated in Table 1 and MM (methyl mercaptan) were enclosed in a Tedlar bag to measure the concentrations of MM and DMDS (dimethyl disulfide) 2 hours and 24 hours immediately after injection of the offensive odors by a gas chromatograph (GC).

The test conditions were defined as follows.

Tedlar bag capacity: 5 L
Initial gas (MM) concentration: 100 ppm
Temperature: room temperature (20 to 25° C.)
Weight of deodorant glass agent: 1 g
Particle size of deodorant glass agent: D50=4.21 μm
Specific surface area of deodorant glass agent: 1.54 m²/g

Operations similar to the above operations were conducted without the deodorant glass agent as a blank.

This test was requested of Environmental Science Laboratory.

### Deodorization Test Method 2 (Artificial Air Atmosphere):

A test similar to the above-described test was conducted in an artificial air atmosphere (oxygen concentration: 20% and nitrogen concentration: 80%).

As is the case with the deodorization test method 1, this test was requested of Environmental Science Laboratory.

### Measurement Result and Consideration:

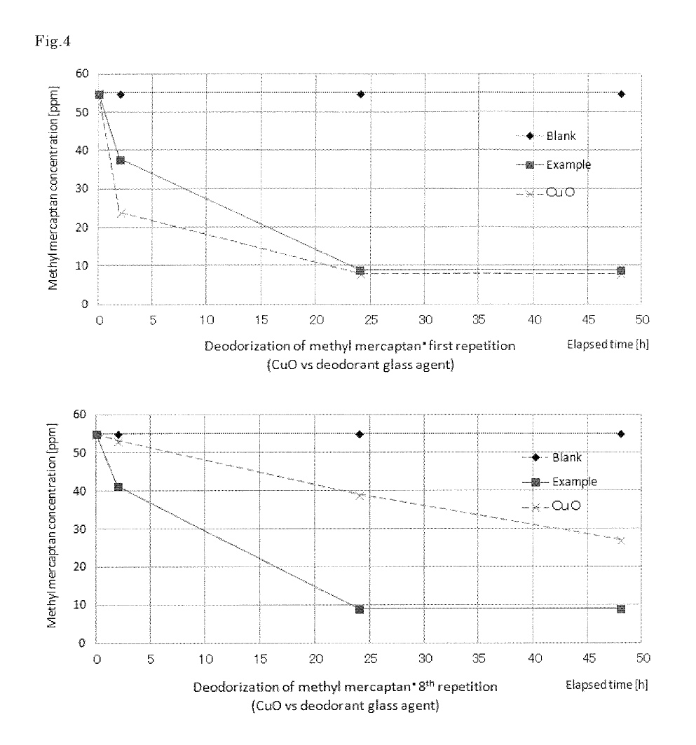

FIG. 2 shows the result of Deodorization test method 1, and FIG. 3 shows the result of Deodorization test method 2.

While DMDS was present from the time point of 0 hour also in the blank, as shown in FIGS. 2 and 3, it was confirmed that DMDS was contained as a contaminant in the gas used. As regards MM→DMDS, although natural oxidation slightly occurs, the deodorant glass agent evidently promotes the production of DMDS as compared with the blank. This reaction involves dimerization of MM into DMDS.

The GC was held up to 90 minutes in order to check the presence of other sulfur components, and, during that, the presence of sulfur components other than MM and DMDS was confirmed, but especially no peaks were confirmed.

If the deodorizing mechanism of the deodorant glass agent is a sulfurization reaction as in the prior art soluble glass agent, the binding between the sulfur component and the copper component would occur. However, not the binding of the sulfur component with copper, but the conversion from MM to another sulfur component DMDS was confirmed as seen in the GC result. The conversion quantity is also considered to be almost equivalent (in consideration, for example, of the reduction in MM in the blank itself).

Also, the deodorizing effect evidently increased due to the presence of oxygen as shown in FIG. 3. The deodorant glass agent is considered to be a catalyst which promotes the MM→DMDS reaction through oxygen. CuO, which is known to exhibit the deodorizing mechanism based on the catalytic action, also promotes an MM→DMDS reaction through oxygen. It is said to be mediated by oxygen adsorbed on its surface. The deodorant glass agent may also exhibit a similar catalytic action. The deodorizing effect, which was confirmed also in the nitrogen atmosphere, might possibly be attributed to oxygen adsorbed on the glass surface before enclosing.

The reaction formula is assumed as follows.

2CH₃—SH+oxidant→CH₃—S—S—CH₃+2H⁺+2e⁻

### Example C: Comparative Test Between CuO and Deodorant Glass Agent

### Deodorization Test Method:

The deodorant glass agent having the glass composition indicated in Table 1 and a CuO reagent, respectively, and MM were enclosed in a Tedlar bag to measure the MM concentration in the bag in accordance with elapsed time by means of a gas detecting tube.

The test conditions were defined as follows.

Tedlar bag capacity: 1 L
Initial gas (MM) concentration: 55 ppm (repeated 8 times at 55 ppm)
Temperature: room temperature (20 to 25° C.)
Weight of deodorant glass agent: 0.1 g
Particle size of deodorant glass agent: D50=4.21 μm
Specific surface area of deodorant glass agent: 1.54 m²/g
CuO: Wako reagent, particle size (value described: 5 μm) and specific surface area: 0.38 m²/g

Operations similar to the above operations were conducted without the deodorant glass agent as a blank.

### Measurement Result and Consideration:

It was confirmed that both of the deodorant glass agent and CuO converge at nearly about 10 ppm in, as shown in FIG. 4. This is an error of the gas detecting tube due to the production of DMDS by the catalytic action. (A sulfur component other than MM, if present, cannot be identified, and thus becomes an error factor.) Simply from the CuO content, the deodorant glass exhibited a high deodorizing effect despite the fact the content of the deodorant glass agent was about 1/10 of that of the CuO reagent.

It was confirmed that the deodorizing speed of CuO was superior at the first repetition, but that the relationship between them was reversed at the 8^{th} repetition, and that the deodorizing speed of the deodorizing glass agent was superior. Specifically, it can be seen that the deodorizing glass agent maintained the deodorizing speed also at the 8^{th} repetition, but that the deodorizing effect of CuO tends to be reduced. CuO is known to be poisoned (catalyst degradation) when deodorizing sulfur-based offensive odors, and the reduction in deodorizing effect is considered to be caused by this. It was confirmed in the present Example that vitrification leads to a stable catalyst state.

### Example D: Comparison Between Soluble Glass Agent and Deodorant Glass Agent=Comparison Between Deodorant Glass Agent Based on Sulfurization Reaction and Deodorant Glass Agent Based on Catalytic Reaction

### Soluble Glass Agent Preparing Method:

### Soluble Glass 1

Typical soluble glass agent containing CuO (IONPURE®) commercial product

### Soluble Glass 2

Magnesium phosphate (94.26 g), 89% by weight of phosphoric acid (157.76 g) and silver oxide (4.0 g) were mixed and held at 300° C. for 3 hours. Next, the dried product of this mixture was molten at 1300° C. for 1 hour to prepare glass having the glass composition indicated in the following Table 2. This glass was pulverized to prepare a sample.

### Soluble Glass 3

Potassium phosphate (71.36 g), primary calcium phosphate (38.05 g), copper oxide (26.17 g) and 89% by weight of phosphoric acid (117.72 g) were mixed and held at 300° C. for 3 hours. Next, the dried product of this mixture was molten at 1300° C. for 1 hour to prepare glass having the glass composition indicated in the following Table 2. This glass was pulverized to prepare a sample.

### Soluble Glass 4

Boric anhydride (12.05 g), sodium nitrate (5.62 g), ultrafine powder silica (product name: SNOWTEX S) (5.26 g), alumina powder (0.2 g), copper chloride (21.4 g) and pure water (60 ml) were stirred with a high-speed stirring machine to prepare a sol. Thereafter, 1 ON ammonia water (3 ml) was added to the sol for gelation. The gel was dried at 120° C. for 180 minutes in a dryer, and then calcined in a calcination furnace at ambient temperature→525° C. for 30 minutes, at 525° C. for 10 minutes, 525° C.→950° C. for 30 minutes, and 950° C. for 30 minutes to prepare a glass agent having the glass composition indicated in the following Table 2. This glass agent was pulverized to prepare a sample.

TABLE 2
Basic compositions of soluble glasses (mol %)

| | Soluble | Soluble | Soluble | Soluble |
|---|---|---|---|---|
| | glass 1 | glass 2 | glass 3 | glass 4 |
| B₂O₃ | | | | 43.8 |
| SiO₂ | | | | 22.1 |
| CaO | | | 10 | |
| Na₂O | 3.0 | | | 8.3 |
| CuO | | | 10 | 25.4 |
| Al₂O₃ | | | | 0.5 |
| Ag₂O | 1.0 | 2 | | |
| P₂O₅ | 49.5 | 49 | 55 | |
| MgO | 46.5 | 49 | | |
| K₂O | | | 25 | |
| Ag₂O amount | 1.12 × 10⁻⁵ | 2.13 × 10⁻⁵ | | |
| in 0.1 g of | | | | |
| sample [mol] | | | | |
| CuO amount | | | 7.87 × 10⁻⁵ | 3.54 × 10⁻⁴ |
| in 0.1 g of | | | | |
| sample [mol] | | | | |
| Particle size | 4.31 | 4.19 | 4.08 | 4.27 |
| (D50) [μm] | | | | |

### Deodorization Test Method:

The deodorant glass agents having the glass compositions indicated in Table 1 and Table 2 presented above, respectively, and hydrogen sulfide were enclosed in a Tedlar bag to measure the hydrogen sulfide concentration in the bag in accordance with elapsed time by means of a gas detecting tube.

The test conditions were defined as follows.

Tedlar bag capacity: 1 L
Initial gas (hydrogen sulfide) concentration: 55 ppm
Temperature: room temperature (20 to 25° C.)
Weight of deodorant glass agent: 0.1 g
Particle size of deodorant glass agent: D50=4.21 vim
Specific surface area of deodorant glass agent: 1.54 m²/g

Operations similar to the above operations were conducted without the deodorant glass agents as a blank.

### Measurement Result and Consideration:

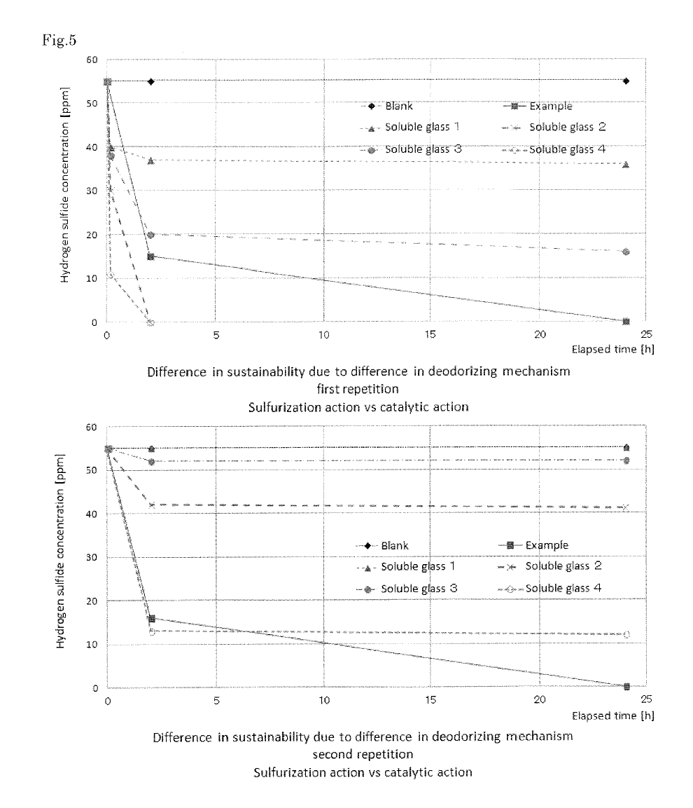

It was confirmed that the soluble glasses had a fast reaction speed because of deodorization based on a sulfurization reaction, as shown in FIG. 5. Therefore, the soluble glass agents were measured also after 10 minutes. Soluble glasses 1 and 3 converged at the first repetition. It was confirmed that the glasses almost arrived at the deodorization limit. Also, these glass agents were confirmed to agglomerate possibly because they have low water resistance and thus easily absorb moisture. The converted values for the amounts of Ag₂O and CuO in the sample amount are indicated as reference values. However, these values represent the total amount of Ag₂O or CuO in the glass agents, and, in fact, Ag₂O or CuO deposited on the surface exhibits deodorizing effect. It is considered that the soluble glasses show a sulfurization reaction on their surface (the discoloration (yellow to brown) supporting the reaction was actually confirmed), and that Ag and Cu contained within the glass do not contribute to the reaction any more. Soluble glass 3 exhibited a slight deodorizing effect also at the second repetition, but agglomerated, and thus there is a possibility that the gas might slowly get into the glass to be deodorized. It was confirmed that the deodorant glass agent is different in deodorizing effect from the soluble glass agents, and thus is more sustainable and provides a larger deodorization amount though having a smaller CuO molar amount than that of Soluble glass 4.

### Example E: Relationship Between CuO Content and Deodorizing Effect

### Deodorant Glass Agent Preparing Method:

After blending of raw materials, the blend was molten at a melting temperature of 1350° C. for 8 hours and poured out, thereby producing glasses having the glass compositions indicated in the following Table 3. Formation after melting was carried out by natural cooling, but can also be carried out by water cooling.

The glass compositions were confirmed by semi-quantitative measurement using a fluorescence X-ray analyzer. The resultant glasses were dry-pulverized in a ball mill and regulated so that D50=4.5 μm or less and D98=50 μm or less by means of a particle size meter. The particles having a particle size (diameter) of 100 μm or more were removed by sieving.

TABLE 3
Compositional ratio of glass (mol %)

| | Experimental | Experimental | Experimental | Experimental | Experimental | Experimental | Experimental |
|---|---|---|---|---|---|---|---|
| | Example 1 | Example 2 | Example 3 | Example 4 | Example 5 | Example 6 | Example 7 |
| B₂O₃ | 15.5 | 15.1 | 15.2 | 14.8 | 14.4 | 13.6 | 13.2 |
| SiO₂ | 55.9 | 56.1 | 54.8 | 54.0 | 53.3 | 52.5 | 52.2 |
| CaO | 7.1 | 6.6 | 6.1 | 6.1 | 5.6 | 5.6 | 5.6 |
| Na₂O | 21.4 | 21.4 | 21.6 | 21.1 | 20.3 | 20.4 | 19.4 |
| CuO | 0.0 | 0.8 | 2.3 | 3.9 | 6.3 | 7.9 | 9.6 |
| Specific | 1.60 | 1.54 | 1.51 | 1.56 | 1.49 | 1.54 | 1.51 |
| surface | | | | | | | |
| area [m²/g] | | | | | | | |
| Particle | 4.03 | 4.25 | 4.33 | 4.17 | 4.39 | 4.21 | 4.32 |
| size (D50) | | | | | | | |
| [μm] | | | | | | | |

### Deodorization Test Method:

The glass agents having the glass compositions indicated in the above Table 3 (deodorant glass agents containing CuO and glass agent containing no CuO) and MM were enclosed in a Tedlar bag to measure the MM concentration in the bag in accordance with elapsed time by means of a gas detecting tube.

The test conditions were defined as follows.

Tedlar bag capacity: 1 L
Initial gas (MM) concentration: 55 ppm
Temperature: room temperature (20 to 25° C.)
Weight of deodorant glass agent: 0.1 g

Operations similar to the above operations were conducted without the deodorant glass agent as a blank.

### Measurement Result and Consideration:

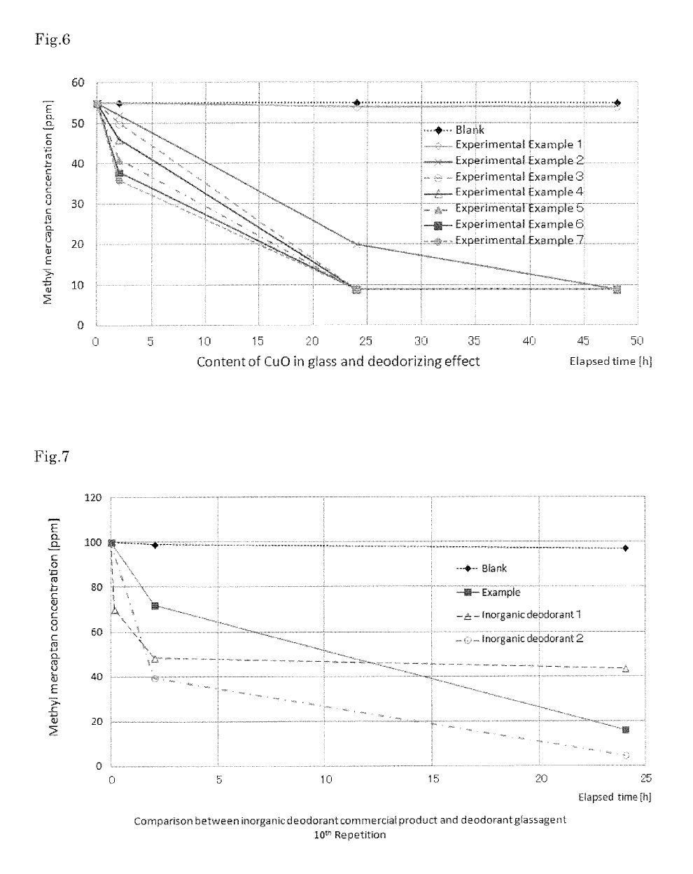

It was confirmed that the deodorizing effects of all of Experimental Examples 1 to 7 which are different in CuO content converge at nearly about 10 ppm, as shown in FIG. 6. This is an error of the gas detecting tube due to the production of DMDS by the catalytic action. (A sulfur component other than MM, if present, cannot be identified, and thus becomes an error factor.)

Also, it was confirmed that, when the glass agents have the same particle size and weight, the deodorizing effect increases (specifically, the deodorizing speed increases) in accordance with the CuO content.

This is caused by increase in CuO content on the glass surface which is contacted with offensive odor in accordance with the CuO content.

However, even Experimental Example 2 having the lowest CuO content deodorizes MM at a high level (55 ppm), and its deodorizing effect is sufficient.

Experimental Example 2 is inferior to Experimental Examples 3 to 7 in terms of deodorizing speed when compared at a time point when 24 hours have elapsed. However, the speed can be easily covered by reducing the particle size and increasing the surface area.

### Example F: Sulfurization Action Associated with Water Resistance and Catalytic Action

The water resistance changes with change in glass composition. At this time, the deodorizing mechanism may possibly change when the glass composition becomes close to that of the soluble glass agent, and thus the Experimental Examples were compared, in the amount of the glass molten, with the typical soluble glass agent IONPURE (Comparative Examples 1 and 2). Comparative Examples 1 and 2 are the typical soluble glass agent “IONPURE (commercial product).”

### Deodorant Glass Agent Preparing Method:

After blending of raw materials, the blend was molten at a melting temperature of 1350° C. for 8 hours and poured out, thereby producing glasses having the glass compositions indicated in the following Table 4. Formation after melting was carried out by natural cooling, but can also be carried out by water cooling.

The glass compositions were confirmed by semi-quantitative measurement using a fluorescence X-ray analyzer. The resultant glasses were dry-pulverized in a ball mill and regulated so that D50=4.5 μm or less and D98=50 μm or less by means of a particle size meter. The particles having a particle size (diameter) of 100 μm or more were removed by sieving. Experimental Examples 8 to 10 were prepared so that the CuO content (mol %) was equivalent.

TABLE 4
Glass compositional ratio (mol %) (fluorescence X-ray semi-quantitative analysis result)

| | Comparative | Comparative | Experimental | Experimental | Experimental |
|---|---|---|---|---|---|
| | Example 1 | Example 2 | Example 8 | Example 9 | Example 10 |
| B₂O₃ | 94.83 | 44.93 | 13.59 | 17.36 | 18.34 |
| SiO₂ | 3.00 | 39.96 | 52.50 | 50.82 | 45.70 |
| CaO | | 0.00 | 5.62 | 2.27 | 1.14 |
| Na₂O | 2.02 | 14.97 | 20.36 | 21.55 | 26.79 |
| CuO | | | 7.93 | 8.00 | 8.03 |
| Ag₂O | 0.15 | 0.14 | | | |
| R₂O + B₂O₃ | 96.85 | 59.90 | 33.95 | 38.91 | 45.13 |
| Specific surface | | | 1.54 | 1.56 | 1.47 |
| area [m²/g] | | | | | |
| Particle size (D50) | 4.48 | 4.35 | 4.21 | 4.19 | 4.44 |
| [μm] | | | | | |
| Amount of glass | 100 | 65.3 | 14.6 | 25.1 | 49.7 |
| molten [%] | | | | | |
| Deodorizing effect | X | X | ◯ | Δ | X |
| by catalytic action | | | | | |

### Method for Confirming Amount of Glass Molten

A sample (0.1 g) was immersed in distilled water (100 mL), and held at room temperature (20 to 25° C.) for 24 hours, and then the amount thereof decreased was confirmed.

### Judging Method

The glass agents were evaluated, under the conditions: Tedler capacity: 1 L and MM concentration: 55 ppm, as follows:

x: those which have arrived at the deodorization limit until after the 8^{th} repetition;

Δ: those which have not arrived at the deodorization limit yet, but have confirmed the reduction in deodorizing speed; and

◯: those which have confirmed to have sustainability even after the 8^{th} repetition.

The specific surface areas and particle sizes of the glass agents in the deodorization test are as indicated in Table 4, and the sample weight is 0.1 g.

### Judgment Result and Consideration:

Although Experimental Examples 9 and 10 were also confirmed to have catalytic action, the sulfurization reaction in ion elution, which is similar to that for the soluble glass, seem to have greatly acted due to insufficient water resistance.

### Example G: Comparison in Performance with Highly Sustainable Inorganic Deodorant (Commercial Product)

### Deodorization Test Method 1 (Evaluation of Sustainability):

The deodorant glass agent having the glass composition indicated in Table 1 and MM were enclosed in a Tedlar bag to measure the MM concentration in the bag in accordance with elapsed time by means of a gas detecting tube.

The test conditions were defined as follows.

Tedlar bag capacity: 1 L
Initial gas (MM) concentration: as indicated in Table 5
Temperature: room temperature (20 to 25° C.)
Weight of deodorant glass agent: 0.1 g
Particle size of deodorant glass agent: D50=4.21 μm
Specific surface area of deodorant glass agent: 1.54 m²/g

A deodorization test similar to the above-described test was conducted by using the inorganic deodorants indicated in Table 5 as deodorants for comparative evaluation. In the meantime, both of these inorganic deodorants are commercialized as highly sustainable inorganic deodorants.

TABLE 5

| | Inorganic deodorant 1 | | Inorganic deodorant 2 | |
|---|---|---|---|---|
| Composition | Amorphous composite | | Copper ion-supported | |
| (main component) | composed of silicon | | zirconium phosphate | |
| | dioxide and metal | | | |
| | oxide | | | |
| Fluorescence X-ray | SiO₂ | 65.3 | P₂O₅ | 41.3 |
| semi-quantitative | ZnO | 25.3 | Cu | 19.3 |
| analysis result | Na₂O | 6.9 | Zr | 36.3 |
| [weight %] | | | | |

| | | |
|---|---|---|
| Specific surface area | 287.04 | 7.90 |
| [m²/g] | | |
| Particle size (D50) [μm] | 3.63 | 0.88 |
| Particle size (D98) [μm] | 7.75 | 2.25 |

Also, a deodorization test similar to the above-described test was conducted without the deodorant glass agent as a blank.

Deodorization Test Method 2 (Condition where Moisture Exists):

The deodorant glass agent having the glass composition indicated in Table 1, inorganic deodorants 1 and 2 in Table 5 and CuO reagent, respectively, MM and distilled water were enclosed in a Tedlar bag to measure the MM concentration in the bag in accordance with elapsed time by means of a gas detecting tube.

The test conditions were defined as follows.

Tedlar bag capacity: 1 L
Initial gas (MM) concentration: 55 ppm
Temperature: room temperature (20 to 25° C.)
Weight of deodorant glass agent: 0.1 g
Particle size of deodorant glass agent: D50=4.21 μm
Specific surface area of deodorant glass agent: 1.54 m²/g
Amount of distilled water added: 500 μl (the entire surface of the sample was wetted)
CuO: Wako reagent, particle size (value described: 5 μm) and specific surface area: 0.38 m2/g

Also, a deodorization test similar to the above-described test was conducted without the deodorant glass agent as a blank.

### Measurement Result and Consideration:

TABLE 6

| | Initial concentration (ppm) |
|---|---|
| First | 80 |
| Second | 70 |
| Third | 56 |
| Fourth | 60 |
| Fifth | 20 |
| Sixth | 55 |
| Seventh | 59 |
| Eighth | 54 |
| Ninth | 73 |

When repetition was conducted 10 times while the initial gas concentration was changed as indicated in Table 6 above, a similar tendency was confirmed until the 10^{th} repetition, as shown in FIG. 7. That is, Inorganic deodorant 1 has a high instant deodorizing effect, but converges because of deodorization limit (adsorption limit). Inorganic deodorant 2 and the Example can deodorize MM at a high level, and the deodorizing speed of Inorganic deodorant 2 is superior when they have the same weight. Inorganic deodorant 1 converges, but can reproduce its deodorizing effect when used to deodorize offensive odor with which MM was replaced (reset). Both of them maintain their deodorizing effect even at the 10^{th} repetition despite the offensive odor present at a high level.

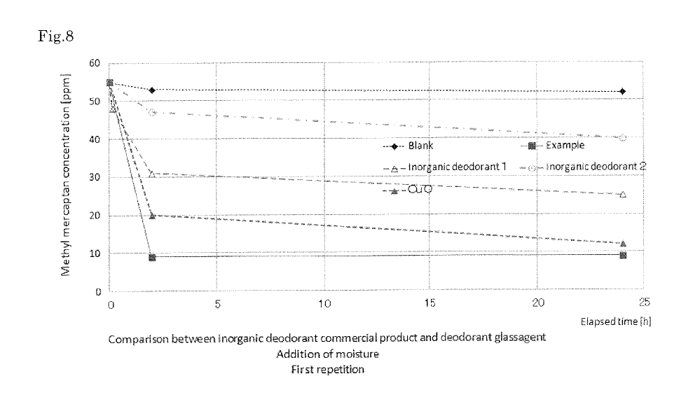

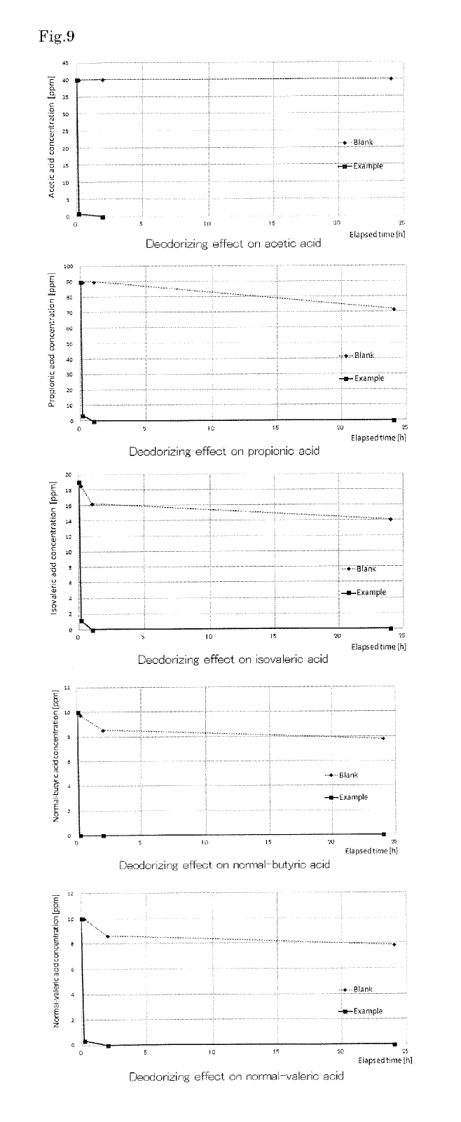

A change in deodorization tendency upon addition of moisture was confirmed, as shown in FIG. 8.

The instant deodorizing effect of Inorganic deodorant 1 was confirmed to be lowered. This is considered to be due to the fact that the instant effect was lowered when its surface gets wet since the agent exhibits high physical adsorption. It was confirmed that Inorganic deodorant 2 can provide no satisfactory deodorizing effect in an environment where moisture exists. In this Example, the addition of moisture was confirmed to considerably improve the deodorizing speed. In this Example, there is a possibility that the presence of moisture would facilitate the catalyst effect and that the deodorizing mechanism based on the sulfurization reaction would be added by ion elution. Since this Example is a highly water-resistant agent, there is a high possibility that the presence of moisture would facilitate the catalyst effect. Also, the result was obtained that the deodorizing speed of the Example was faster than that of CuO even at the first repletion under the moisture addition condition (see FIG. 4).

In the meantime, the concentration was slightly lowered, but was not confirmed to be clearly reduced, in the blank. This result suggests that MM is not dissolved in water, and that the deodorizing effects of the respective agents could be evaluated.

### Example H: Test for Confirming Deodorizing Effect on Lower Fatty Acids

### Deodorization Test Method:

The deodorant glass agent having the glass composition indicated in Table 1 and offensive odor were enclosed in a Tedlar bag to measure the offensive odor concentration in the bag in accordance with elapsed time by means of a gas detecting tube.

The test conditions were defined as follows.

Tedlar bag capacity: 1 L
Temperature: room temperature (20 to 25° C.)
Weight of deodorant glass agent: 0.1 g
Particle size of deodorant glass agent: D50=4.21 μm
Specific surface area of deodorant glass agent: 1.54 m²/g

Operations similar to the above operations were conducted without the deodorant glass agent as a blank.

### Measurement Result and Consideration:

The deodorant glass agent was confirmed to have deodorizing effect on acetic acid, propionic acid, normal-butyric acid, normal-valeric acid, isovaleric acid and any lower fatty acids.

### Example I: Test for Confirming Deodorizing Effect on Trans-2-Nonenal

### Deodorization Test Method:

The deodorant glass agent having the glass composition indicated in Table 1 and CuO reagent, respectively, and trans-2-nonenal were enclosed in a Tedlar bag to measure the offensive odor concentration in the bag in accordance with elapsed time by means of a high-speed liquid chromatograph.

In the high-speed liquid chromatographic method, the gas within the bag was collected in a DNPH cartridge, and a DNPH derivative was eluted by passing acetonitrile through this cartridge. Then, the resultant eluate was measured by a high-speed liquid chromatograph to calculate the concentration of the gas within the bag.

The test conditions were defined as follows.

Tedlar bag capacity: 4 L
Temperature: room temperature (20 to 25° C.)
Weight of deodorant glass agent: 0.1 g
Particle size of deodorant glass agent: D50=4.21 μm
Specific surface area of deodorant glass agent: 1.54 m²/g
CuO: Wako reagent, particle size (value described: 5 m) and specific surface area: 0.38 m²/g

Also, operations similar to the above operations were conducted without the deodorant glass agent as a blank.

This test was requested of Environmental Science Laboratory.

### Measurement Result and Consideration:

TABLE 7
(unit: ppm)

| | |
|---|---|
| Classification of | Elapsed time |

| samples | 0 min | 30 min | 2 h | 6 h |
|---|---|---|---|---|
| Analyte | 19 | 9 | 9 | 8 |
| Control product | 19 | 13 | 12 | 10 |
| Blank test | 19 | 18 | 16 | 16 |

The deodorizing effect on trans-2-nonenal was confirmed as indicated in Table 7.

1. A deodorant comprising a glass, the glass containing:
46 to 70 mol % of SiO₂;
15 to 50 mol % of B₂O₃ and R₂O (R=Li, Na, K) in total;
0 to 10 mol % of R′O (R′=Mg, Ca, Sr, Ba);
0.1 to 23 mol % of CuO; and
0 to 3.5 mol % of Al₂O₃.

2. The deodorant according to claim 1, wherein the glass containing:
5 to 20 mol % of B₂O₃; and
10 to 30 mol % of R₂O (R=Li, Na, K).

3. A deodorant comprising a glass, the glass containing:
50 to 63 mol % of SiO₂;
23 to 44 mol % of B₂O₃ and R₂O (R=Li, Na, K) in total;
2 to 7 mol % of R′O (R′=Mg, Ca, Sr, Ba);
1 to 13 mol % of CuO; and
0 to 3.5 mol % of Al₂O₃.

4. The deodorant according to claim 3, wherein the glass containing:
8 to 18 mol % of B₂O₃; and
15 to 26 mol % of R₂O (R=Li, Na, K).

5. A deodorant comprising a glass, the glass containing:
51 to 55 mol % of SiO₂;
12 to 16 mol % of B₂O₃;
19 to 22 mol % of Na₂O;
4.5 to 6.5 mol % of CaO;
4 to 13 mol % of CuO; and
0 to 3.5 mol % of Al₂O₃.
